(19) United States
(12) Patent Application Publication
(10) Pub. No.: US 2008/0312552 A1
(43) Pub. Date: Dec. 18, 2008

(54) Method to detect change in tissue measurements

(76) Inventors: Qienyuan Zhou, Del Mar, CA (US); Pak-Wai Lo, San Diego, CA (US); Koenraad A. Vermeer, Voorburg (NL)

Correspondence Address: STALLMAN & POLLOCK LLP, 353 SACRAMENTO STREET, SUITE 2200, SAN FRANCISCO, CA, 94111, US

(21) Appl. No.: 12/157,850

(22) Filed: Jun. 13, 2008

Related U.S. Application Data
(60) Provisional application No. 60/936,066, filed on Jun. 18, 2007.
(60) Provisional application No. 60/962,911, filed on Aug. 1, 2007.

(51) Int. Cl. A61B 3/00

(52) U.S. Cl. 600/558

(57) ABSTRACT

The present invention relates to the detection of statistically significant changes in tissue characteristics within the eye. Change in a tissue characteristic is statistically significant when the magnitude of the change exceeds the test-retest measurement variability. One embodiment of the present invention analyzes the data using more than one statistic in order to capture global, regional, and/or local changes that are essential to clinical interpretation of changes in a tissue characteristic. In one embodiment of the present invention, the tissue characteristic tested is RNFL thickness.

### PRIORITY

This application claims the benefit of the filing date under 35 U.S.C.§ 119(e) of Provisional U.S. Patent Application Ser. No. 60/936,066, filed on Jun. 18, 2007 and Provisional U.S. Patent Application Ser. No. 60/962,911, filed on Aug. 1, 2007, which are hereby incorporated by reference in their entirety.

### TECHNICAL FIELD

The subject invention relates to the detection of statistically significant changes in the topography of a structure within the eye. Of particular interest are changes in the eye determined by optical measurements of the retinal nerve fiber layer (RNFL). More specifically, an approach is described where the thickness of the RNFL is evaluated using at least two different analysis techniques in order to improve diagnostic accuracy. Improved methods for displaying the results are also disclosed.

### BACKGROUND

Accurate assessment of RNFL thickness makes early detection and better management of glaucoma possible. Traditionally, glaucoma is monitored by testing for loss of vision. By the time vision loss is detected, a significant amount of nerve fiber may have already been compromised. In contrast, using recently developed optical instruments, structural damage to the RNFL can be detected before field vision loss is detectable. Early detection enables early treatment and improved outcomes. RNFL damage is highly correlated with a structural diagnosis of glaucoma.

Several modem devices can provide a measure of RNFL thickness. The assignee herein markets the GDx™ scanning laser polarimeter, which measures the retardance of the RNFL using a polarimetry technique. The measured retardance is proportional to the RNFL thickness. The assignee also markets the Stratus OCT™ and Cirrus™ HD-OCT retinal imagers which use Optical Coherence Tomography (OCT) to measure the RNFL thickness.

While these devices have provided clinicians with improved tools for detecting glaucoma, there is a continuing need for sensitive and reliable detection of glaucomatous progression. Glaucoma progression happens slowly. Early detection of degradation in the RNFL or visual function enables earlier and more effective medical intervention, improving visual function outcomes. The subject disclosure is directed to a number of improvements in data analysis algorithms, integration of the analyses, and display techniques which facilitate the early detection of disease progression. These improvements can be implemented using any instrument which obtains spatial measurements of structures within the eye or functions of the eye that can then be analyzed in accordance with the subject invention.

### SUMMARY

The present invention is defined by the claims and nothing in this section should be taken as a limitation on those claims. Advantageously, embodiments of the present invention overcome the above-described problems in the art and provide analysis techniques and displays improving diagnostic accuracy.

In one aspect of the subject invention, tissue data are obtained over at least two visits. The data are evaluated to determine if there has been a statistically significant change in a characteristic of the tissue between the visits. More than one type of analyses are used in combination to improve the accuracy of the evaluation.

In another aspect of the subject invention, tissue data are obtained over at least three visits. Tissue data may be topography data, it may be tissue thickness data, or it may be data descriptive of other tissue characteristics.

In another aspect of the subject invention, tissue changes are parameterized into global, regional and local measures for a multi-modal change detection method.

In another aspect of the subject invention, the tissue data are RNFL measurement data. RNFL measurement data obtained over at least two visits are evaluated using more than one type of analyses to determine if there has been a statistically significant loss in RNFL thickness.

In another aspect of the subject invention, techniques are developed to improve accuracy of RNFL change detection, including detecting/excluding blood vessel and ONH regions, employing dual baselines, and confirming RNFL loss with additional follow-up visit.

In another aspect of the subject invention, when multiple scans per visit are available for analysis, individual-based test-retest variability is applied to identify patient-specific statistically significant RNFL loss.

In another aspect of the subject invention, when individual-based test-retest variability cannot be assessed due to lack of repeated measurements per visit, population-based test-retest variability is applied to identify statistically significant RNFL loss.

In another aspect of the subject invention, certain display techniques have been developed to convey to the clinician the most relevant aspects of the analysis. In one aspect, the display color codes regions of concern using fundus image overlays. In another aspect, the display color codes significant change in the TSNIT plots based on regional analysis. In another aspect, trend charts display the statistical significance of the progression of the disease based on global analysis and the rate of RNFL loss to facilitate assessment of clinical significance of the detected progression.

The analysis of the change over time is very important in determining disease progression. The detection of RNFL change is very important in determining glaucomatous progression. A reliable change detection method and a comprehensive and easy-to-understand report are therefore extremely desirable, for both the clinicians and the patients. The subject invention meets a long-felt and unsolved clinical need.

### BRIEF DESCRIPTION OF THE DRAWINGS

FIG. 1 is an exemplary overview of GDx Change Analysis for detecting a significant change in RNFL thickness.

FIG. 2 illustrates the cluster size assessment with blood vessel exclusion in regional analysis.

FIG. 3 illustrates the cluster size assessment with blood vessel exclusion in local analysis.

FIG. 4 is an exemplary flow diagram of a Change From Baseline (CFB) method for detecting a significant change from baseline measurements.

FIG. 5 illustrates a report displaying multi-modal change detection results with inter-instrument measurements.

FIG. 6 is an exemplary flow diagram of a Statistical Image Mapping (SIM) method for detecting a significant change.

FIG. 7 illustrates a report displaying multi-modal change detection results.

FIG. 8 illustrates a report displaying trends before and after clinical intervention.

FIG. 9 illustrates a report identifying quality issues.

FIG. 10 illustrates a report displaying risk of disability for various life milestones.

FIG. 11 illustrates a report displaying risk of disability with multiple data types and axes.

### DETAILED DESCRIPTION

It should be understood that the embodiments, examples and descriptions have been chosen and described in order to illustrate the principles of the invention and its practical applications and not as a definition of the invention. Modifications and variations of the invention will be apparent to those skilled in the art. The scope of the invention is defined by the claims, which includes known equivalents and unforeseeable equivalents at the time of filing of this application. While the description herein relates primarily to thickness and topographic measurements of the retina, the subject invention can be applied to other measurements tissue characteristics structures within the eye. While the tissue characteristics described herein are primarily acquired and stored by a GDx™ scanning laser polarimeter, these tissue characteristics could alternatively have been acquired by any of various alternative devices, including, but not limited to, the Stratus OCT® ophthalmic imager, Visante® OCT ophthalmic imager, Cirrus™ HD-OCT ophthalmic imager, or various other devices. The embodiments, examples and descriptions chosen to describe and illustrate the principles of the invention and its practical applications will, for the most part, be based on application of the invention to polarimetric RNFL measurements acquired with the GDx™ scanning laser polarimeter, in particular the GDx VCC and its successors. Modifications and variations of the invention will be apparent to those skilled in the art.

Change in a tissue characteristic is statistically significant when the magnitude of the change exceeds the test-retest measurement variability [8]. Relatively small changes in retinal thickness extending over a large area are clinically relevant because they may provide an early indication of glaucoma. Even a small change in thickness, consistent over a large area, is readily detectable by a statistically reproducible global parameter such as an average thickness derived from a large number of independent measurements. Statistically significant changes may be either global or regional in nature and are differentiated by the scope of their support. On the other hand, large changes in retinal thickness, even if limited to a relatively small area, are also clinically relevant. Analysis of localized parameters, which inherently exhibit higher measurement variability, nominally detects large changes over small regions. Therefore, in accordance with the subject invention, it is desirable to analyze the data using more than one statistic in order to capture global, regional, and/or local changes that are essential to clinical interpretation of changes in a tissue characteristic. In particular, analyses of more than one statistic measuring global, regional and/or local change in tissue thickness is of immense clinical value in interpreting and predicting glaucomatous progression.

Summary parameters, such as average TSNIT, or parameters averaged over a global region of interest, such as TSNIT averaged over superior or inferior quadrants, exemplify parameters used in global change detection. Global change detection looks at a measure over a broad region and identifies change over the region as a whole. Global change detection is used to identify relatively small levels of change over the entire measurement area (or a substantial portion of the entire measurement area). Statistically, global detection can detect smaller changes than local or regional detection.

Regional change detection identifies change over regions smaller than the entire field of view, such as over clusters of pixels. Sectional measurements about the optic nerve head (ONH), such as the TSNIT plot, exemplify parameters used in regional change detection. Statistically, regional detection is used to detect smaller changes in depth than local detection but requires larger changes than needed by global detection. Regional change detection provides sensitivity and selectivity with respect to changes in size and changes over area where neither global nor local detection are well suited.

Local parameters, such as pixel-by-pixel measurements of RNFL thickness, exemplify parameters used in local change detection. Localized change detection detects changes over measurement points or pixels. Statistically, local detection requires larger changes for detection than regional or global detection. Nominally, local change detection compares RNFL image measurements about the optical nerve head (ONH). Local detection is associated with early indicator of glaucomatous pathology such as wedge defects. Frequently a wedge is a segment of an annular ring, however, the term may also apply trapezoidal or even nearly rectangular shape. The term “wedge” generally refers to a region commonly wider further from the ONH and narrower nearer the ONH, but generally applies to other regions of limited scope.

In one instance, global, regional and local change detection are performed through an event-based and population-based algorithm (Change-From-Baseline (CFB)) [8]. In another instance, global, regional and local change detection are performed through a trend-based and individual-based algorithm (Statistical non-Parametric Mapping (SnPM) or Statistical Image Mapping (SIM)) [8]. CFB detects change based on a triggering event of RNFL reduction in follow-up visits. SIM analyzes the trend of the RNFL measurements and detects statistically significant trends of RNFL loss. These algorithms were used elsewhere prior to this invention; however, novel and non-obvious changes have been made to improve the performance of the algorithms for change detection. In particular, the combination of multi-modal tests is novel and central to one aspect of our invention.

Since it is desirable to perform change detection across different instruments, both the CFB and SIM have been modified to handle change detection on inter-instrument measurements while retaining specificity.

In order to improve the accuracy of change detection, areas obscured by blood vessel as well as areas within the ONH can be excluded in Change Analysis.

Use of more than one baseline visit can provide a more robust baseline reference for comparison with the follow-up visits and reduces the likelihood of false alarm detection for CFB based analyses.

An inter-visit confirmation approach can be employed to reduce the likelihood of false alarm detection. Such an inter-visit confirmation approach requires changes detected the first time in a parameter to be confirmed in a subsequent visit for the same parameter.

In one aspect of the invention, a comprehensive change detection report is designed to display and summarize the multi-modal RNFL change detection results. The detection report communicates the multi-modal change detection results in a simple and clinically meaningful way. This report is particularly useful for the doctor or examining practitioner, but can also be a valuable tool for communicating with the patient or care provider. The report provides a summary of the multi-model change analysis. One such report contains detailed information of the quality of the measurement data, display images (local analysis) and TSNIT plot (regional analysis) with areas of statistically significant change highlighted in colors, provides trend charts of the summary parameters (global analysis) with statistically significant change highlighted in colors. Importantly, this report provides an assessment of the rate of the RNFL loss.

The multi-modal change detection of RNFL measurements is important for monitoring and detecting progression of glaucoma. In glaucoma progression detection, global, regional and local changes each provide diagnostically useful information for the treatment and monitoring of the disease. Each of these detection modes can be clinically informative individually, but they can also be synergized to improve sensitivity of overall change detection. A comprehensive change detection report provides a vehicle to synergize the information of said detections.

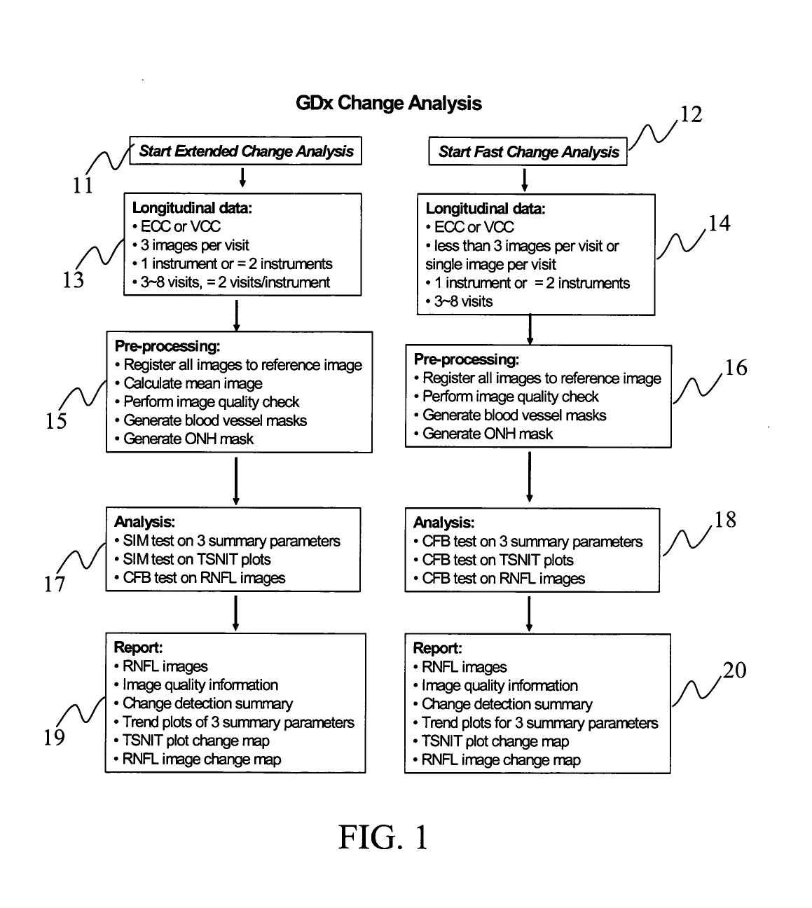

FIG. 1 is an overview of the multi-modal RNFL change detection method. The two (2) methods of change detection analyses illustrated are: Extended Change Analysis and Fast Change Analysis. Fast Change Analysis and Extended Change Analysis have been developed to analyze different data types. Both Extended Change Analysis (also called Extended Analysis) and Fast Change Analysis (also called Fast Analysis) follow the same general process steps, but differ in some individual step implementations. The general steps are: locating the longitudinal data to be analyzed, preprocessing the located datasets, analyzing the data, and reporting the analysis results. Since individual step implementation differs, the first decision in Change Analysis is determining whether to proceed to Start Extended Change Analysis 11, or Start Fast Change Analysis 12. As indicated in 13, Extended Analysis requires three (3) or more images per visit for the data to be analyzed. Fast Analysis requires one (1) or more images per visit 14. Aside from the difference in the number of images per visit, steps 13 and 14 are similar in that they identify the data type based on imaging mode, number of measurements per visit, number of instruments used in data collection, and number of visits included in the data collection set. The next step is preprocessing the data. Preprocessing in 15 and 16 includes performing spatial registration of images from all visits, performing image quality check on each image used, and detecting blood vessels and ONH in the images to generate the blood vessel and ONH masks. Extended Analysis utilizes three (3) or more images per visit; hence, a meaningful statistical variance to a mean image can be estimated and preprocessing 15 calculates a mean image in Extended Analysis. The next step is to perform multi-modal change analysis for each of the three (3) data types, namely, summary parameters, TSNIT Plot, and RNFL image. For Fast Analysis, CFB is always chosen 18; for Extended Analysis, SIM is chosen to analyze summary parameter and TSNIT plot data 17, while CFM is chosen to analyze RNFL image data 17. Finally, the last step of the Change Analysis is report generation. The report is similar in layout for both Extended Analysis 19 and Fast Analysis 20. This report is an important vehicle that synergizes the results of the multi-modal analysis and delivers a simple and clinically relevant summary. The report contains RNFL images, image quality information, change detection summary, trend plots, TSNIT change map, and RNFL change map, whenever available.

The particular algorithm selection is not an essential part of the subject invention. Alternative algorithm selections may achieve similar performance. For example, SIM may be employed in all three (3) modes in Extended Analysis and CFB may be employed for all three (3) modes in Extended Change Analysis as well. As will be understood by those versed in the art, other algorithms distinguishing or identifying change may be used as well.

The CFB method compares the difference between follow-up visits and the baseline visits to a measure of the reproducibility. In Fast Analysis, the measure of reproducibility is set to a fixed value. On the other hand, in Extended Analysis, the measure of reproducibility is determined based on the repeated measurements of the test eye.

The SIM method is based on the assumption that in the absence of change, a measure of change should be insensitive to random permutations of the measurements. If change is present, the observed order of measurements yields a value that is more extreme than the values in most of the permutations. In one embodiment, the measure of change is defined as the ratio of the slope (measurement value versus time) of linear regression and its standard error. Alternatively, the measure of change may be any measure describing the trend information of the data.

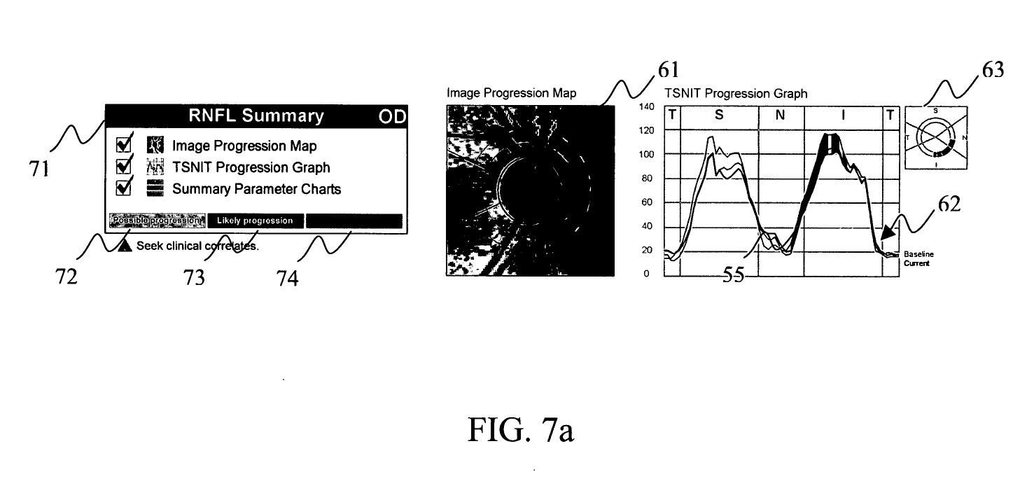

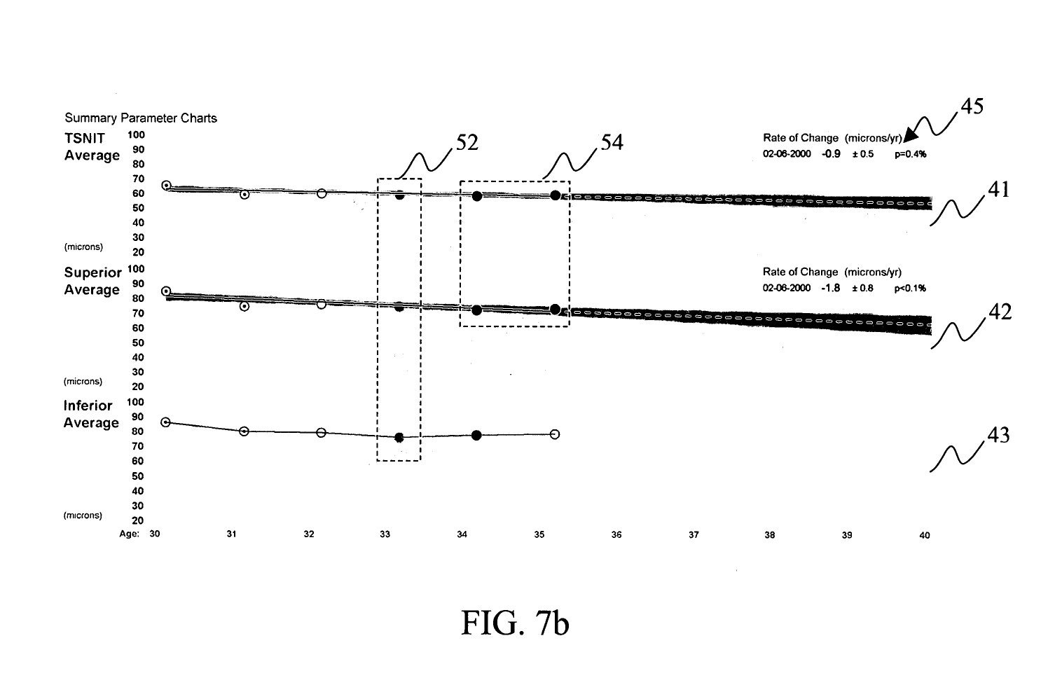

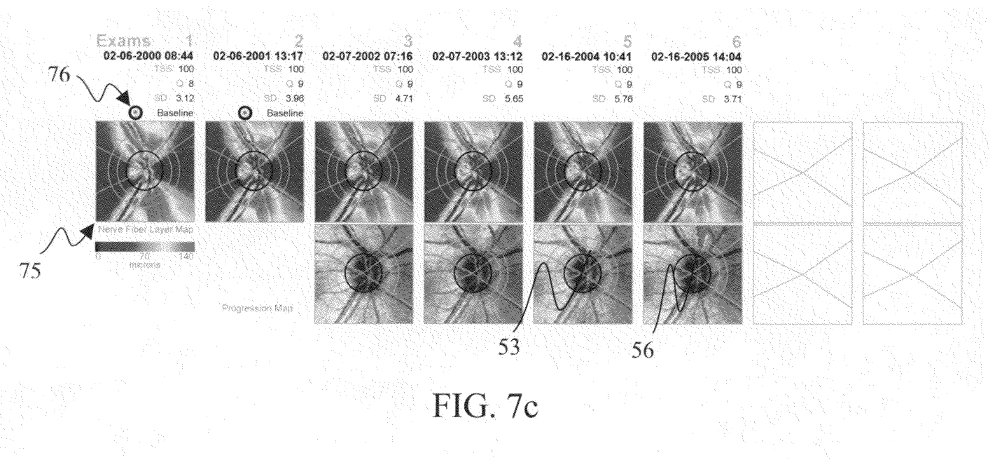

A clear and accurate message is useful at the conclusion of the multi-modal analysis. In one embodiment, if a change is detected for the first time in a parameter, it is labeled as “Possible” change; if such change is confirmed in a consecutive visit, it is labeled as “Likely” change. The particular naming is not an essential part of the subject invention. Alternative clinically useful terminology may achieve similar benefit. For example, a change detected for the first time can be labeled as “Change” and change confirmed in a consecutive visit can be labeled as “Confirmed Change”. For consistency, “Possible” change and “Likely” change will be used hereinafter. (See FIG. 7, discussed below in greater detail.)

The integration of the multi-modal analysis is such that if “Likely” change is detected in any one of the multi-modal measures, “Likely” RNFL change is reported for the test eye; if only “Possible” change is detected in one or more measures, “Possible” RNFL change is reported for the test eye; if neither “Likely” or “Possible” change is detected in any of the measures, “No change detected” is reported for the test eye. Alternative integration logic may be applied. For example, when three or more multi-modal measuring techniques are used and a high priority is set for eliminating false alarms, the report may require that two measuring techniques agree before a “Possible” or “Likely” change is declared. Alternatively, if the sensitivity of the various techniques are different or vary, a probabilistic result may be reported.

An analysis change report summarizes the results of the multi-modal analysis and integration.

The statistical analyses employed in the multi-modal change detection are based on the CFB-based algorithm and the SIM-based algorithm. The CFB algorithm has been used in opthalmology to detect topographic changes on and around the ONH (such as the approach described by Chauhan and adopted by the optical instrument manufacturer Heidelberg in their Heidelberg Retina Tomograph (HRT) imaging device). The SIM algorithm has been used in the field of radiology and opthalmology to detect change (such as the approach described by Patterson). However, separate and significant modifications to these prior art methods (discussed-below in the following five (5) paragraphs) are required to improve sensitivity and specificity of multi-modal change detection developed herein.

Topographic Change Analysis (TCA) for topographic measurement of the optic nerve was published in 2000 by Chauhan et al. The CFB approach herein is similar to the TCA approach in that they are both event detection based on change from baseline. Four key differences between the Chauhan TCA and our CFB follow.

1) CFB is based on two (2) baselines and TCA is based on one (1) single baseline. CFB two-baseline approach is based on the important observation that inter-visit test-retest variability plays a key role in the measurement variability assessment, in addition to the intra-visit test-retest variability (the proposed dual baselines approach helps to improve the progression detection specificity in the presence of inter-visit variability).

2) The CFB approach herein has been extended from individual-based change analysis to include population based change analysis so that longitudinal data series with only one (I) measurement per visit can also be analyzed with this approach. This extends the approach to cases where individual test-retest variability is not available.

3) CFB developed herein makes clear distinction between intra- and inter-instrument measurements and applies the appropriate test-retest variability accordingly.

4) Finally, for the multi-modal analysis to detect both diffuse and local loss, the cluster size threshold for different modes are selected based on a preferred clinically meaningful size and then the threshold for the significance level is selected accordingly to achieve the desired specificity. This distinguishes the method from the prior art references [1-3] which first selected the threshold for the significance level and then the detection size, which usually rendered detection size immaterial to clinical use. The relationship between the significance level threshold and the detection size threshold are investigated in Vermeer et al [6].

SIM was introduced into opthalmology for topographic image change analysis by Patterson et al in May 2005. The technique was well known in the field of radiology for a much longer time. Our implementation of SIM has significantly deviated from the initial approach reported by Patterson et al. The key differences include: 1) in order to detect change in TSNIT plot with the SIM approach, the algorithm is modified to account for the spatial characteristics of test-retest variability; and 2) SIM developed herein makes clear distinction between intra- and inter-instrument measurements and applies different regression model accordingly.

The SIM method described in the referenced Patterson article employs a three-step approach to find an area showing change. In the first step, each data point is evaluated individually and converted to a probability score (p-value). The second step thresholds these points, and determines the maximum size of the resulting clusters. By repeating this for different permutations, each cluster size would be associated with a probability score and the area statistic can then be determined. The third step determines the area statistic of the observed order of measurements and compared to those obtained in step two. A change would be detected if the observed area statistic (from the observed order of the measurements) were smaller than a set percentage of those generated in the different permutations from step two. However, this approach only works well if the noise in the data points is not strongly correlated. For instance, if the noise is spatially fully correlated, either all measurement points, or none at all, would show change exceeding the selected p-value threshold while converting each data point into a probability score in step one. This would result in large area statistics for many permutations and would render detection of small area of loss impossible because a small area of loss has a small area statistic.

One instance of the subject invention solves said problem by scaling the p-values of the data points in step one to incorporate data from other spatial location(s) of interest, such as neighboring pixels in a 2-D image or neighboring points in a 1-D data series. Information from such data points would be used to determine the scaling for each p-value. This scaling helps reduce the impact of the spatially correlated noise.

The p-values may be scaled in various ways. The scaling should be such that the most extreme change in the data points corresponds to the most extreme p-values (e.g. p=0 or p=1) and neutral values (e.g. p=0.5) should remain neutral or nearly neutral. For a linear scaling, the mathematical relationship (for each point) between the unscaled p-values and the scaled p-values is:

P_{scalcd}=(P_{unscaled}−0.5)·w+0.5

In this equation, w specifies the scaling factor with values between 0 and 1. If w=0, all values are transformed to neutral p-values (p=0.5). For w=1, the scaled p-values will exactly match the unscaled ones. The scaling factor w incorporates information of the entire data set to be analyzed. For example, in the regional analysis, the entire TSNIT plots would be used to provide scaling for each individual TSNIT plot.

The relative slope of each point may be used to determine the scaling factor. Alternatively, the ratio between the slope and the standard error of the measurements can also be used.

### Preprocessing

In FIG. 1, preprocessing occurs in both analysis paths in 15, and 16. Preprocessing of the longitudinal data prepares the data for change analysis. Preprocessing may includes reference image selection, image registration, image quality check, mean image calculation, and blood vessel mask and ONH mask generation. Registering images to a reference image aligns measurements for comparison. Precomputed image statistics can improve image quality checks as well as improve data comparison from images of different means. Performing a quality check on images enables weighting image data and/or elimination of unreliable data. Masking out blood vessels and the optic nerve head also removes sources of errors from unreliable data.

In the FIG. 1 process, the reference image is chosen to be the comparison base for other images from the same eye. For image alignment, other images are aligned to the reference image. The reference image may be a single image automatically selected by software. The automatic selection may be made from a collection of single images of the same eye, with the image with the highest image quality score selected to be the reference image. Alternatively, the user can directly select the reference image and forego the software selection. Alternatively, the reference image can be based on a combination of single images, such as a mean image, or a feature extraction from a single image, etc.

In the FIG. 1 process, it is advantageous for the user to review both the ONH ellipse and the macular circle placements on the reference image. The ONH ellipse should properly outline the optic disc margin because the size of the ONH ellipse is important for the ONH mask generation. For best performance, the macular circle is centered on the fovea. Preferably, the ONH ellipse and macular circle placements in the reference image are duplicated after image registration on all other images within the comparison set. This improves consistency and saves time. Alternatively, ellipse and circle placements can be reviewed in each individual image and the combination of said individual placements can then be used on the images after registration.

In the FIG. 1 process, image registration is performed based on fundus images for all measurements from the same eye. Image registration works on measurements acquired with a single instrument and/or with multiple instruments and provides transformations for spatial corrections. The spatial corrections may include horizontal and vertical translations, rotation, horizontal and vertical magnification, and shear effect. In one embodiment, the registration is performed with sub-pixel accuracy using sub-pixel interpolation. Alternatively, other spatial correction accuracy may be used to reduce variability introduced by spatial misalignment.

In the FIG. 1 process, fundus locations occupied by retinal blood vessels as well as locations within the optic disc are excluded in the change analysis. This is achieved through the combination of blood vessel (BV) and optic nerve head (ONH) masks [6]. BV mask is a composite BV pattern generated from the BV patterns of single images in the longitudinal data series. ONH mask is based on the ONH area defined in the reference image. Alternatively, additional mask can be used to remove image artifact, such as saturated pixel(s), border pixel(s), etc. Similarly, the BV and ONH mask can be slightly expanded in all directions to ensure complete exclusion.

In some instances, said masks may be applied to regional and local analysis and not to global analysis. In other instances, said masks can be applied to all type of multi-modal analysis.

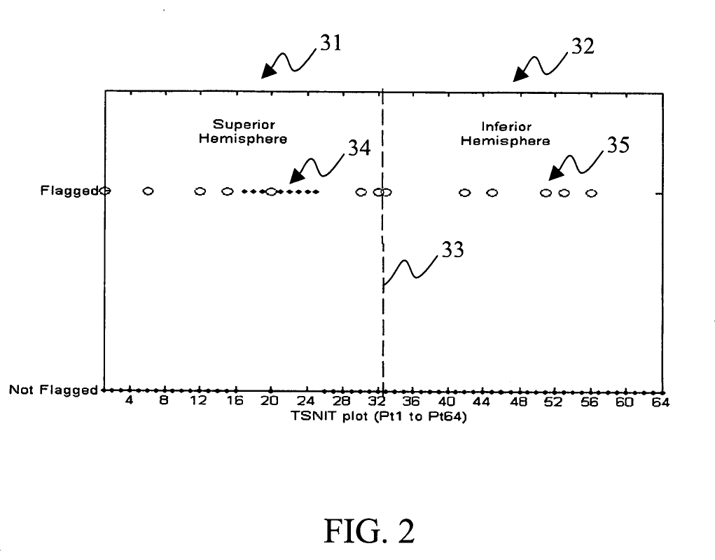

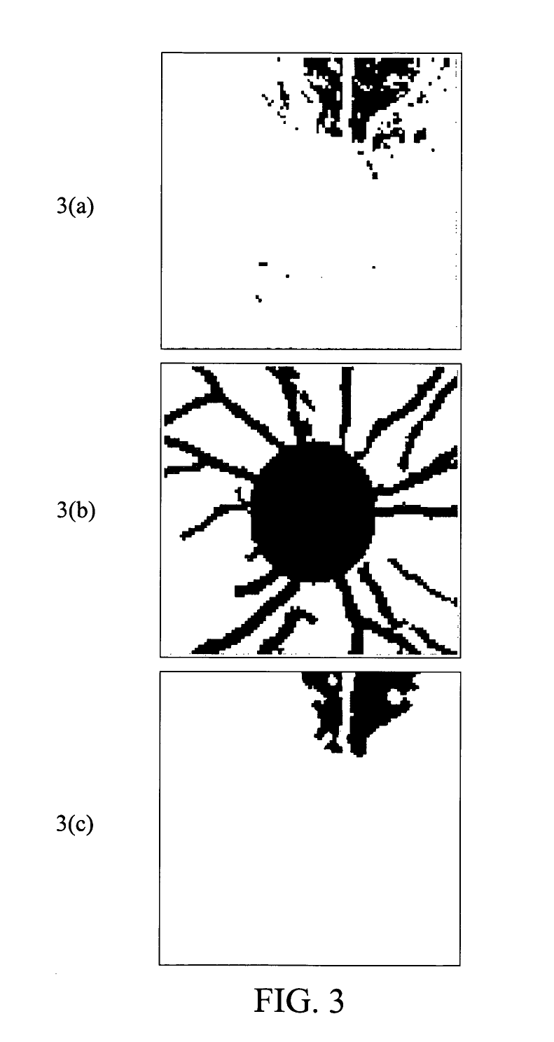

Masking blood vessels may leave objectionable holes in the data field that are problematic or inconvenient for later analysis. For this reason, data points on either side of a blood vessel may be connected for analysis. For example, in FIG. 2, TSNIT plot points on either side of a blood vessel are recognized and combined into one (1) cluster, instead of two (2) distinct clusters separated by blood vessel, in the estimation of cluster size analysis. In the regional analysis, the TSNIT plot points are separated into two (2) regions 33, namely, the superior hemisphere 31 and the inferior hemisphere 32. Data points are shown as solid dots 34, and the blood vessel points are shown as circles 35. In FIG. 2, in the superior hemisphere 31, three (3) data points are flagged, followed by a blood vessel points, and then five (5) more flagged TSNIT plot points. Normally, the three (3) connected flagged points and the five (5) connected flagged points would be considered two (2) distinct clusters and a threshold of a minimum of six (6) connected points would not include either cluster in this case. Since these two (2) groups are separated by a blood vessel, they should have been considered as one unit with eight (8) connected flagged points and the same threshold should be able to detect this connected cluster. Another way of presenting this is shown in FIG. 3. The ONH and blood vessels are shown in FIG. 3(b). A raw collection of unfiltered flagged image points is shown in FIG. 3(a). FIG. 3(c) illustrates the processed version of FIG. 3(a). Image points on either side of a blood vessel are recognized and combined in the estimation of cluster size. The separated and distinct flagged image cluster points of FIG. 3(a) are connected with the corresponding blood vessel pattern of FIG. 3(b) to form one (1) distinct cluster shown in FIG. 3(c) [8].

In FIG. 1 pre-processing 15, a mean image is created by averaging each single image within the same visit after image registration. The single images are from the same eye with the same imaging mode (either VCC or ECC) and in the same visit.

In FIG. 1 Extended Change Analysis Pre-processing 15 and Fast Change Analysis Pre-processing 16, an image quality check is performed prior to Extended Analysis 17 or Fast Analysis 18. Image quality check may include checking the image quality of a single measurement, checking an image registration quality metric, and/or checking registration parameters. The user is alerted to a poor quality image in the report or on the examination viewing screen.

### Fast Analysis

In the FIG. 1 process, Fast Analysis provides change detection for longitudinal data series with two (2) or more visits. In one embodiment, Fast Analysis performs analysis on data acquired from three (3) to eight (8) visits, with each visits consisting of single images or a mixture of single and mean images. Alternatively, Fast Analysis can also provide change detection in inter-instrument longitudinal data series.

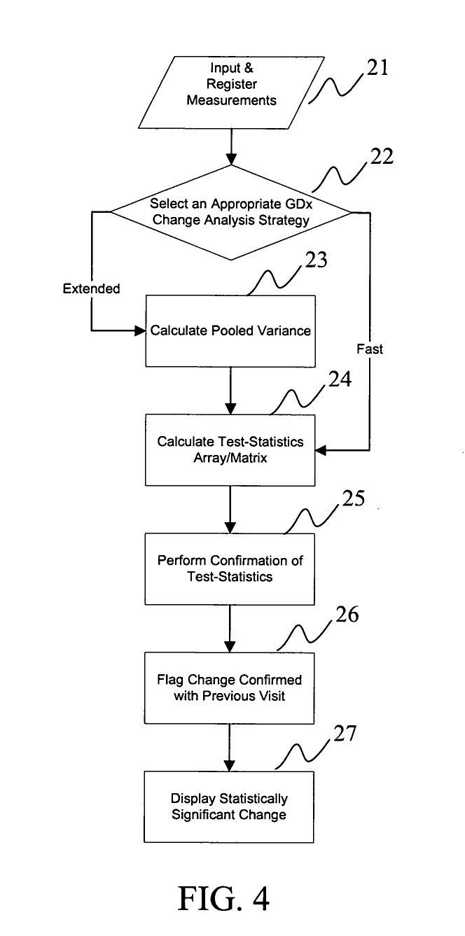

In the FIG. 1 process, Fast Analysis uses the Change From Baseline (CFB) algorithm to analyze change. CFB is applied to all modes of the multi-modal analysis—global (summary parameters), regional (TSNIT plot) and local (RNFL image). FIG. 4 is a flow diagram of the CFB architecture. There are six (6) steps.

(1) obtaining input measurements and performing registration,

(2) selecting a Change Analysis strategy (Fast Analysis or Extended Analysis),

(3) calculating test-statistics for analysis,

(4) performing confirmation of test-statistics,

(5) flagging change confirmed with previous visit, and

(6) displaying said statistically significant change.

The first step in CFB is to obtain and register measurements 21. The first decision in CFB is to select the appropriate Change Analysis strategy 22, depending on the number of measurements per visit. Fast Analysis can be selected for any number of measurements per visit. Alternatively, Fast Analysis is performed when there is one (1) or two (2) measurement per visit. In another aspect of the subject invention, two (2) baseline visits and a minimum of one (1) follow-up visit are required for CFB analysis. The next step is to calculate the test-statistics for the analysis 24. The test-statistic (t) between a follow-up visit and a baseline visit is defined as the difference between the measurements. The next step is performing confirmation of test-statistics 25. Thresholds specific to image mode, number of confirmation test and inherent test-retest variability (intra- or inter-instrument) are used to determine statistically significant change. Negative change is detected when t is less than such thresholds; positive change is detected when t is greater than such thresholds. When there are three (3) visits, two (2) possible test-statistics are calculated (t₃₋₁—test-statistics between the follow-up visit and the first baseline visit; and t₃₋₂—test-statistics between the follow-up visit and the second baseline visit) and such test-statistics are combined on a 2-out-of-2 principle to confirm the change(s) detected in each test-statistic. Similarly, when there are four (4) or more visits, four (4) possible test-statistics are calculated (t₃₋₁—between first follow-up and first baseline; t₃₋₂—between first follow-up and second baseline; t₄₋₁—between second follow-up and first baseline; and t₄₋₂—between second follow-up and second baseline) and such test-statistics are combined to confirm the change(s) detected in each test-statistics. In one embodiment, for four (4) or more visits, the confirmation is based on a 3-out-of-4 (75%) principle. Alternatively, the confirmation can be 2-out-of-4 (50%) principle to enhance sensitivity. Similarly, the confirmation can be 4-out-of-4 (100%) principle to enhance specificity. This utilization of the double-baseline visits is different from the prior art method [4] where the double-baseline visits are averaged to create one (1) single baseline for comparison. The next step is confirming change with previous visit 26. The intra-visit change(s) 25 is/are further confirmed with intra-visit change(s) 25 from previous visit. For instance, if a change is detected in one visit, but the same change is not confirmed in subsequent visit, then no change is detected. On the other hand, if a change is detected in one visit and again confirmed in subsequent visit, it is likely that change has occurred. Such inter-visit confirmation combines change(s) from sequential visit(s) and helps increase the accuracy of detection. The last step of the CFB scheme is displaying said intra-visit and inter-visit confirmed change(s) 27.

In the FIG. 1 process, three (3) summary parameters are used in CFB global change detection. Alternatively, other numbers of representative global measures can be used. Change is detected when at least one of the summary parameters is flagged as changed.

In one embodiment, sixty-four (64) TSNIT plot points are used in CFB regional change detection. Alternatively, other numbers of representative regional measures can be used. CFB is performed on each individual TSNIT plot point as described above. Flagged point(s) on either side of the blood vessel point(s) is/are connected (FIG. 2). In another aspect of the subject invention, points in the upper and lower hemisphere are not connected. In another aspect of the subject invention, TSNIT plot points are flagged when the connected points cluster exceed a meaningful threshold. Such meaningful threshold can be three (3) TSNIT plot points corresponding to approximately seventeen (17) degree sector. Alternatively, other meaningful thresholds can be selected. The event of change occurs when at least one cluster exceeding the cluster threshold is flagged.

CFB may use a region of interest on a 2D image measurement as the basis for local change detection. The region of interest can be of any meaningful size, but is generally larger than 5% of the total, with either individual pixels or super-pixels as the basis unit. A measurement point coinciding with blood vessel area is not used for calculation. CFB is performed on each individual measurement point as described above. As shown in FIG. 3, flagged points on either side of the blood vessel point(s) are connected. Points in the upper and lower hemisphere are not connected. Measurement points are flagged when the connected points cluster exceed a meaningful threshold. Such meaningful threshold can be one hundred fifty (150) points corresponding to approximately a 0.33 mm area. Alternatively, other meaningful thresholds can be selected. The event of change occurs when at least one cluster exceeding the cluster threshold is flagged.

### Extended Analysis

Extended Analysis provides change detection for longitudinal data series with two (2) or more visits. In the FIG. 1 process, Extended Analysis performs analysis from three (3) to eight (8) visits, with each visits consisting of three (3) or more single measurements. In another embodiment, Extended Analysis using SIM also provides change detection in inter-instrument longitudinal data series with a minimum of two (2) visits per instrument. The limit of 8 visits is only a hardware limitation for this embodiment. There is no algorithmic limit.

SIM is the algorithm of choice for the Extended Analysis process of FIG. 1 and is applied to all three (3) multi-modal analysis—global (summary parameters), regional (TSNIT plot) and local (RNFL image). In one embodiment, Extended Analysis uses the SIM approach in the global and the regional analysis and use the CFB approach in the local analysis. Alternatively, CFB can be applied in global and regional analysis while using SIM in local analysis. The principle illustrated hereinafter is apparent to different combination of CFB and SIM approaches.

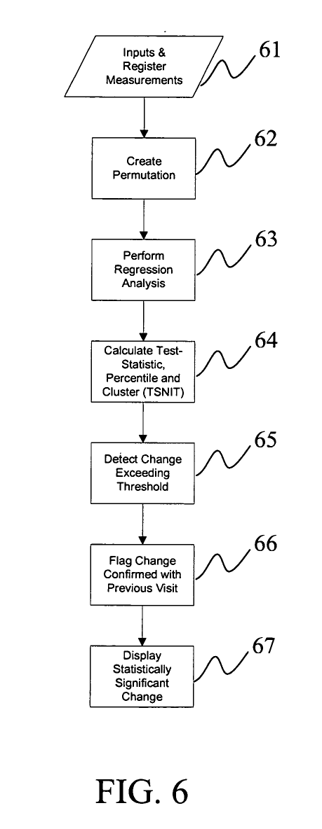

FIG. 6 is an overview of the SIM architecture. There are seven (7) steps, namely, obtaining input measurements and performing registration, creating unique and distinct permutation, performing regression analysis, calculating test-statistic, p-values and cluster, detecting change exceeding threshold, confirming detected change with previous visit, and displaying statistically significant change.

The first step in SIM is to obtain all measurements from each visit for all visits and perform image registration 61. The next step is creating permutations 62. Adequate number of unique and distinct permutations is performed to obtain a good distribution of trend information. The next step is performing regression analysis 63. In one embodiment, linear regression is used for the regression analysis. Alternatively, higher order of regression model can also be used. The next step is calculating test-statistic, p-values and cluster 64. In one embodiment, test-statistic t is defined as the slope of the linear regression model divided by the standard error of the slope. Alternatively, other relative measure of the trend information can be used as the test-statistic for SIM. For inter-instrument data, an offset is added to the regression model to preserve a continuous slope across all visits. A distribution of said test-statistics is obtained and is converted into p-values for statistical comparison. The next step is detecting change exceeding a threshold 65. The test-statistic from the observed order of measurements is then compared to the populations of test-statistics obtained above from the permutations. A change is detected when the test-statistic of the observed order exceeds a desired threshold. The next step is confirming detected change with previous visit 66. Change detected from one (1) visit is confirmed with change in the subsequent visit. For instance, if a change is detected in one visit, but the same change is not confirmed in a subsequent visit, then no change is detected. On the other hand, if a change is detected in one visit and again confirmed in subsequent visit, it is likely that change has occurred. This confirmation approach helps increase the accuracy of detection. The last step of the SIM scheme is displaying the confirmed change(s) in an integrated report 67.

In the FIG. 1 process, three (3) summary parameters are used in SIM global change detection. Alternatively, other number of representative global measures can be used. Change is detected when at least one of the summary parameters is flagged as change.

In one embodiment, sixty-four (64) TSNIT plot points are used in SIM regional change detection. Alternatively, other numbers of representative regional measures can be used. SIM is performed on each individual TSNIT plot point as described above. In one instance, the test-statistic used is defined as the slope of the regression model divided by a smoothed version of the standard error to reduce noise. The p-values converted from the test-statistics may be scaled by a weight factor using trend information of all TSNIT plot points (discussed supra). As shown in FIG. 2, flagged points on either side of the blood vessel point(s) are connected. Individual flagged points (not occurring in pairs) may be extrapolated to the boundary. In this instance, points in the upper and lower hemisphere are not connected. A connected flagged TSNIT plot point cluster is evaluated for each permutation to generate a population of flagged TSNIT plot point clusters. The scaled p-value from the observed order of measurement is then compared to the population of flagged TSNIT point cluster (area statistic) and change is detected if the p-value exceeds a desired threshold. For example, if the p-value falls below the 5^{th} percentile of the permuted population, then a change is detected. Alternatively, other reasonable thresholds can be used. In some instances, thresholds as diverse as the 2^{nd} percentile or 1^{st} percentile may be used, while in other cases, sensitivity and specificity thresholds indicate a choice of even higher thresholds.

CFB is performed on each individual measurement point as described in the CFB section. The same CFB approach for local analysis discussed in the Fast Analysis section can be applied in the local analysis in the Extended Analysis. CFB may use a region of interest on a 2D image measurement as the basis for local change detection; the region of interest can be of any meaningful size and is based on either individual pixels or super-pixels; and measurement points coinciding with blood vessel area is not used for change analysis. The Extended Analysis CFB local analysis different from the Fast Analysis CFB local analysis in two (2) ways. First, Extended Analysis CFB uses mean images for comparison. Second, test-statistic in Extended Analysis CFB is defined as the mean difference between the follow-up visit and a baseline visit divided by the square root of the pooled intra-visit variance of all visits up to the visit of interest (23 and 24 in FIG. 4).

Similarly, using said two differences, Extended Analysis CFB can be applied to other modes of multi-modal analysis, such as global and regional analysis. Alternatively, the same Extended Analysis SIM can be applied to local analysis on image measurements with the same scaled p-value and cluster analysis approaches.

### Estimate Rate of Change

When change is detected in a longitudinal data series, it is important to estimate the rate of change to facilitate assessment of clinical significance. In one embodiment, the rate of change is provided for global analysis (for summary parameters) and implementation is similar for both Fast Analysis and Extended Analysis. While the implementation may only covers global analysis, similar trend analysis can be implemented for regional and local analysis and the trend information can be presented in a table, a plot, or an image format. The following description focuses on the implementation for global analysis.

In one aspect of the subject invention, the output of the trend analysis is based on linear regression; the slope, 95% confidence intervals of the slope, and the p-value significance of the slope are reported. Alternatively, other trend information can also be displayed, such as confidence intervals of the slope at different significance level, prediction intervals of the slope, relative trend information, so forth and so on. For inter-instrument data series, the linear regression model includes an offset parameter between measurements from different instruments to accommodate instrument bias. Alternatively, nonlinear regression may be applied if warranted by data quality and expected model behavior.

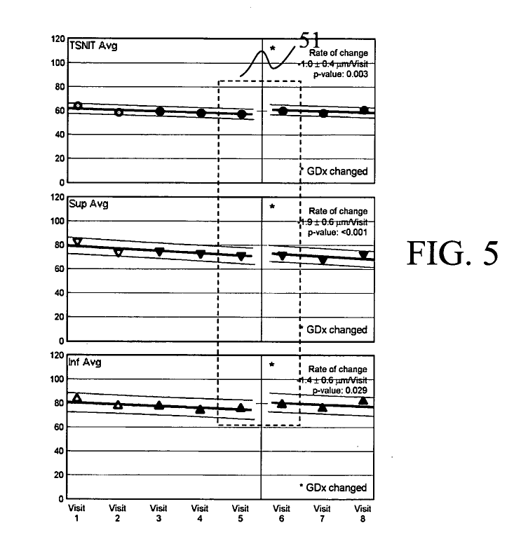

In another aspect of the subject invention, positive trends are differentiated from negative trends and a statistically significant trend is plotted along with the 95% prediction or 95% confidence intervals, or other desirable significance levels. A clear display of a trend line and prediction intervals for inter-instrument data series can reveal instrument induced measurement variation and other inter-instrument data characteristics. FIG. 5 illustrates an example of such trend plot. The trend is broken between the visits 51 when instrument-swap takes place. Such plots also provide useful information regarding the magnitude of bias between instruments.

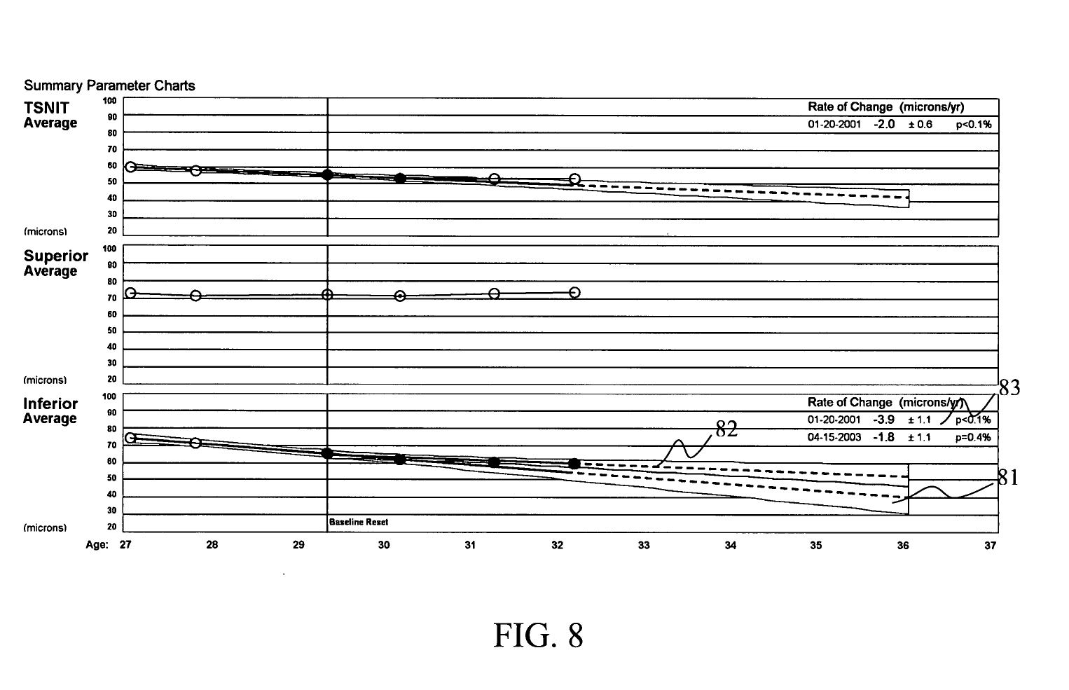

In another aspect of the subject invention, dual trend analysis is implemented to facilitate the comparison of a trend before and after clinical intervention. FIG. 8 shows an example of a dual trend representation. The follow-up duration is divided into two periods and the rate of change is assessed for each period respectively, with the rates displayed on a common display. Trends before clinical intervention 81 and after clinical intervention 82 are displayed. Alternatively, the follow-up duration may be divided into more than two periods and the rate of change assessed and displayed for each period. Such trends conveniently indicate the effectiveness of treatment and pinpoints the impact on each of the global measures or measure(s) of interest.

### Inter-Visit Confirmation

Inter-visit confirmation improves change analysis specificity. Implementation of the inter-visit confirmation is similar for both Fast Analysis and Extended Analysis.

Change detection differentiates between a change that is first detected (“Possible” change) and a change that is confirmed with a consecutive visit (“Likely” change) within each test parameter.

For summary parameters in the global analysis, if negative change is detected one time in any parameter, such change is labeled as “Possible loss”. If negative change is detected in two consecutive visits for the same parameter, such change is labeled as “Likely loss”. If positive change is detected at any time in a parameter, such change is labeled as “Possible increase”.

For the TSNIT plot in regional analysis, the same rules of inter-visit confirmation for the parameters apply. Additionally, for the TSNIT plot to be labeled as “Likely loss”, the cluster size of the confirmed change should exceed a predetermined meaningful cluster threshold on two consecutive visits. Confirmed change compares clusters in the same location. Cluster thresholds can be the same threshold used in the CFB regional analysis of three (3) TSNIT plot points cluster. Alternatively, other desired cluster size of interest can be used.

The same rules for the inter-visit confirmation for the parameters apply in the local analysis of an RNFL image. Additionally, for the RNFL image to be labeled as “Likely loss”, the cluster size of confirmed change should exceed a predetermined meaningful cluster threshold in two consecutive visits in the same locations. Such threshold can be the same threshold as in the CFB local analysis, one-hundred-fifty (150) pixels cluster. Alternatively, other desired cluster size of interest can be used.

The exact labeling of the detected change is not important to the subject matter of the invention. Other meaningful labels can also be used to signify change detected at one time and change detected and confirmed with subsequent visits.

### Multi-Modal Analysis Integration

Integration of the multi-modal analysis is provided through a change analysis summary. Implementation is similar for both Fast Analysis and Extended Analysis. Such a summary of change analysis integrates the detection results of each of the three (3) modalities.

If “Likely loss” is detected in at least one (1) of the three (3) modalities (global, regional, and local), the summary of the change analysis would conclude “Likely loss” is detected. If no “Likely loss” is flagged and “Possible loss” is flagged in at least one (1) of the three (3) modalities, the summary of the change analysis would conclude “Possible loss”. If neither “Likely loss” nor “Possible loss” is flagged, the summary of the change analysis would conclude “no loss detected”. Alternatively, an integrative conclusion of such change analysis can be combined from a weighted sum of each modality. Other integration techniques are possible, especially when additional modalities are present. Depending on decision criteria for sensitivity and specificity additional tools known to those versed in the art of decision theory can be applied. If a sufficient model is available, fuzzy logic reasoning may be applied. Alternative rule based decisions can be used to alter control false positives or false negatives.

The integration stage is an important step to achieve sensitivity to different shapes (diffuse, focal, or other morphological shapes) and different depth of loss with the multi-modal change detection. The design philosophy is that different modes in the change detection are tuned to be more sensitive to different shapes and depth of change, and therefore, it is not necessary for a change to be detected in more than one mode for the eye to be flagged as changed. Alternative design philosophies may combine different modalities with different sensitivities and may then require an integration stage requiring multi-modal detection to flag change.

### Change Analysis Report

A comprehensive change detection report is designed to display and summarize the multi-modal RNFL change detection results. The detection report is instrumental in communicating the multi-modal change detection results to the doctor and the patient in a simple and clinically meaningful way.

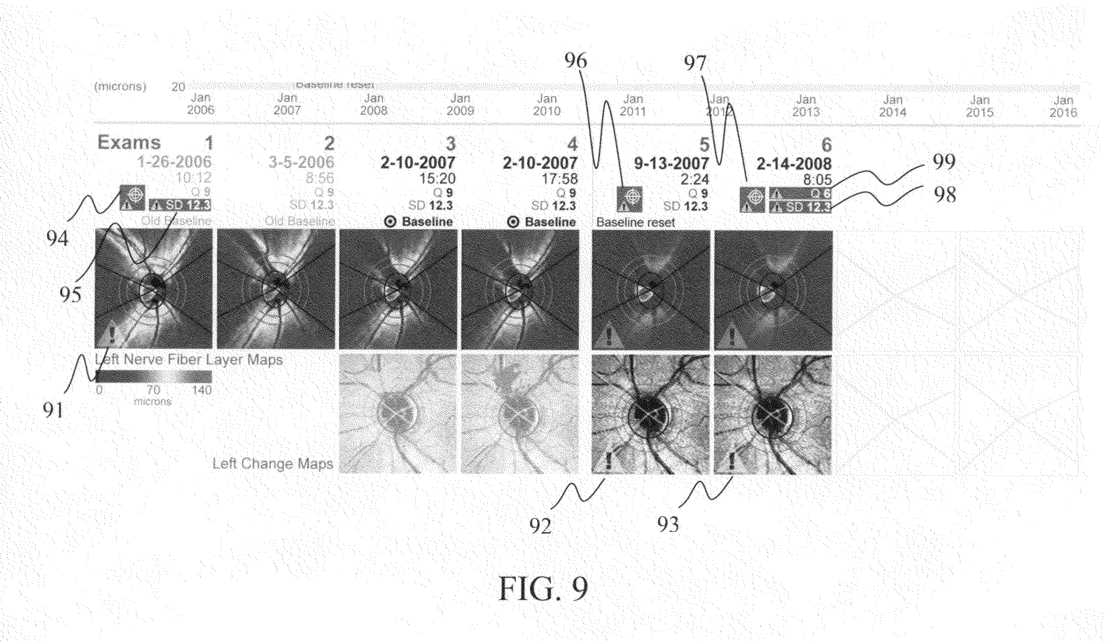

One format for the report is shown in FIG. 7. In this format, the results of the three (3) change detection modes are displayed in the same report. The integration of the multi-model analysis is provided through the summary box 71. In this example, there is a check mark for each of the three (3) modes for “Likely” progression and the summary of the change detection for the eye is labeled as “Likely progression” accordingly. The image change map 61 displays the result of the RNFL image based local analysis, the TSNIT change graph 62 displays the result of the TSNIT plot based regional analysis, and the summary parameter charts (41, 42 and 43) displays the results of the parameter based global analysis. A small icon 63 to the upper-right corner of the TSNIT change graph displays the angular locations of the change relative to the center of the ONH. The RNFL images of the data series and history of change based on the localized analysis are displayed 75, along with important image quality information for each visit (FIG. 9). The baseline visits are clearly marked 76 on top of the corresponding RNFL images. The colors in the report indicate the state and direction of change; gray for no loss detected (no progression detected), yellow for first detection of loss (Possible progression) 72, red for confirmed detection of loss (Likely progression) 73, and purple for possible increase 74. The same color scheme is applied to all three (3) modes of analysis and the summary box 71. Possible progression is displayed in yellow in the global parameters 52, and the local parameters 53. Likely progression is displayed in red for the global parameters 54, the regional parameters 55 and the local parameters 56.

FIG. 9 illustrates said change detection report in case of quality issue in the measurement data. When data quality issue is detected in one or more of the visits, a warning icon (!) is displayed (91, 92 and 93). Warning icon and symbols representing different quality issues are also displayed along the images of the visits exhibited the quality issue. The symbols indicate Visit 1 shows image registration issue 94 and exhibits higher than usual test-retest variability 95; Visit 5 shows image registration issue 96, and Visit 6 exhibits image registration 97, test-retest variability 98, and image quality 99 issues.

The report provides a summary of the multi-modal change analysis, offers detailed information of the quality of the measurement data, displays images (local analysis) and TSNIT plot (regional analysis) with areas of statistically significant change highlighted in colors, furnishes trend charts of the summary parameters (global analysis) with statistically significant change highlighted in colors, and importantly, presents assessment of the rate of the RNFL loss.

As shown in FIG. 7, the summary parameter charts serve two (2) purposes: displays the results of the global analysis, TSBIT Average 41, Superior average 42 and inferior average 43, through color coded data points corresponding to individual visits and display the rate of change both graphically and numerically 45. The multi-modal analysis report further supports trend analysis of two (2) follow-up periods as illustrated in FIG. 8. This is to provide both numerical 83 and graphical (81 and 82) comparison of trends before and after clinical intervention for easy assessment of treatment efficacy.

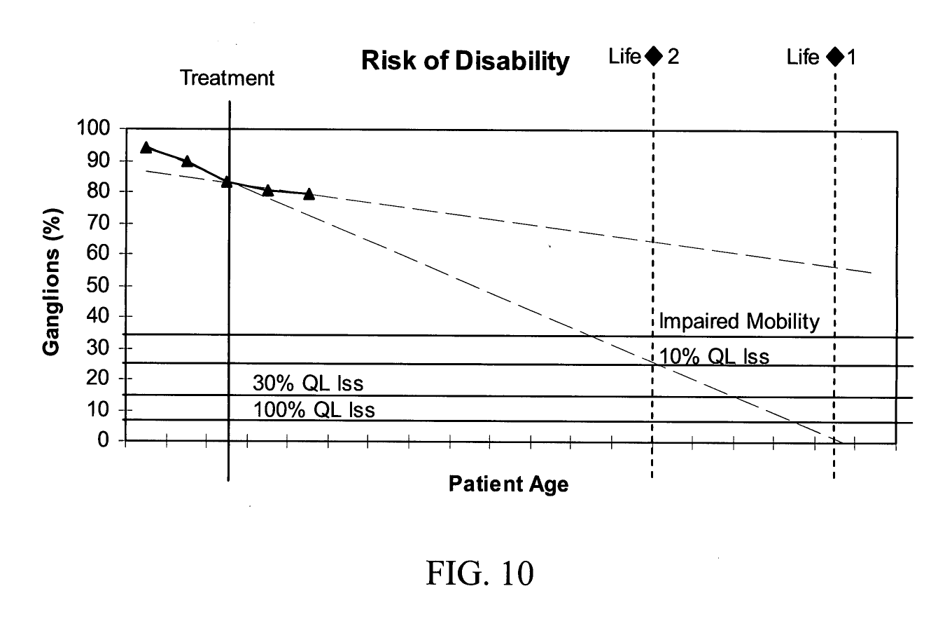

FIG. 10 illustrates an alternative trend analysis display. This display presents the measurement data and trend prediction, but also incorporates Treatment Milestones and Life Milestones as well as predicted quality of life level markers, here related to the percentage of ganglion cells properly functioning. Quality of life markers might also be clinical measures of visual function, such as are presently measured by perimetry, or to metrics that are based upon one or more structural measurement and one or more functional measurement and one or more metabolic measurement and one or more risk factor estimate. Alternatively, the quality of life level markers could be based on RNFL thickness or any other measure of the tissue characteristic that can be correlated to ability to see or other quality of life criteria (like ability to drive or read). The quality of life level markers could, alternatively, be considered a measure of disability.

The Life Milestones may be specific ages, dates, or actuarial estimates. Actuarial estimates such as 50th percentile life expectancy or 95th percentile life expectancy or any other statistically stratified or not statistically stratified life expectancy estimate, e.g. statistical life expectancy percentile estimates based upon the specific medical status of the particular patient under consideration, perhaps based upon blood analysis or genetics or other medical index. Their purpose is to highlight the expected impairment that a predicted trend predicts at a particular Life Milestone. This display highlights the risk versus reward attributes for one or more treatments (or lack of treatment). The trend need not be a linear prediction, but may be a higher order polynomial or other modeled trend.

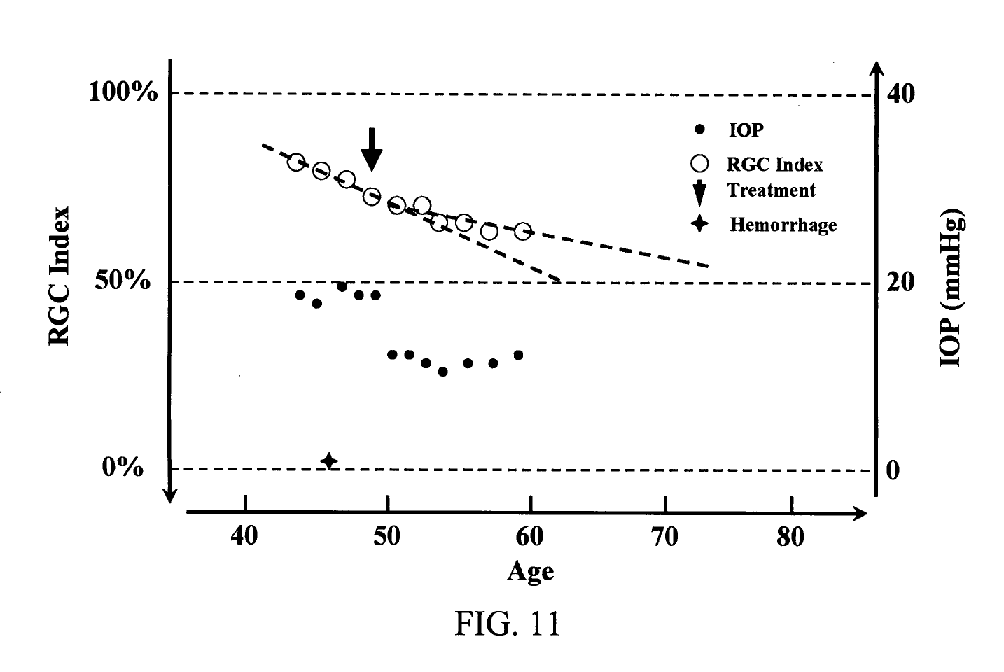

FIG. 11 illustrates another alternative trend analysis display. Again, the display presents measurement data, trend prediction, and a Treatment Milestones. This display also shows two types of data; one is a measure of retinal tissue and the other is a measure of intraocular pressure. Two vertical axes are presented and labeled, scaling the two types of data. Also, a Major Event is shown, here a disc hemorrhage. The disc hemorrhage is an indication of risk for further progression. Treatment has an immediate affect on IOP and a delayed, yet significant, effect on the rate of loss of in the retinal ganglion cell (RGC) Index.

It should be understood that the embodiments, examples and descriptions have been selected and described in order to illustrate the principles of the invention and its practical applications and not as a definition of the invention. The subject invention can be applied to other topographical structures imaged using other imaging modalities. Such structures and modalities include, but are not limited to: RNFL thickness maps or optic nerve head (ONH) topography acquired using an Optical Coherence Tomography (OCT) device, ONH topography acquired using a fundus imager such as a confocal scanning laser opthalmoscope, or corneal topography measured using OCT or ultrasound. Modifications and variations of the invention will be apparent to those skilled in the art. The scope of the invention is defined by the claims, which includes known equivalents and unforeseeable equivalents at the time of filing of this application.

The following references are incorporated herein by reference:
[1] Chauhan et al. Technique for Detecting Serial Topographic Changes in the Optic Disc and Peripapillary Retina Using Scanning Laser Tomography. Invest. Opthalmol. Vis. Sci., Vol. 41, No. 3, March 2000.
[2] Chauhan et al. Optic Disc and Visual Field Changes in a Prospective Longitudinal Study of patients With Glaucoma. Arch Opthalmol. 2001; 119:1492-1499.
[3] Chauhan, The Essential HRT Primer, Chapter 5: Detection of Glaucomatous Changes in the Optic Disc, Heidelberg Engineering, On-line publication
[4] Brochure: Humphrey® Glaucoma Progression Analysis™ (GPA™) Software—An advanced approach to monitoring disease progression, Carl Zeiss Meditec, Inc. (2003)
[5] Patterson et al. A New Statistical Approach for Quantifying Change in Series of Retinal and Optic Nerve Head Topography Images. Invest. Opthalmol. Vis. Sci., Vol. 46, No. 5, May 2005.
[6] Vermeer et al. Modeling of Scanning Laser Polarimetry Images of the Human Retina of Progression Detection of Glaucoma. IEEE Trans. Medical Imaging, Vol. 25, No. 5, May 2006.
[7] Anderson D R, Drance S M, eds. Encounters in glaucoma research 3. How to ascertain progression and outcome. Amsterdam: Kugler, 1996:184-186.
[8] Zhou et al., “Progression Analysis Algorithms for GDx VCC Retinal Nerve Fiber Layer Measurements”, Arvo Abstract (2006).

What is claimed is:

1. A method of facilitating the identification of a statistically significant change in the characteristic structure of tissue within the eye of a patient comprising:
(a) obtaining measurements acquired during at least two separate visits;
(b) evaluating the results of the measurements to identify a change in tissue characteristics occurring between the measurements, said evaluation including at least two different types of analyses, said two different analyses being selected from the group consisting of:
(i) a global analysis of a tissue characteristic;
(ii) a regional analysis of a tissue characteristic; and
(iii) a local analysis of a tissue characteristic; and
(c) displaying or storing the results of the at least two different types of analyses

2. A method of facilitating the identification of a statistically significant change in the topography of a structure in the eye of a patient comprising:
(a) obtaining measurements acquired during at least two separate visits;
(b) evaluating the results of the measurements to identify a topographical change occurring between the measurements, said evaluation including at least two different types of analyses, said two different analyses being selected from the group consisting of:
(i) a global analysis of topography;
(ii) a regional analysis of topography; and
(iii) a local analysis of topography; and
(c) displaying or storing the results of the at least two different types of analyses.

3. A method as recited in claim 2, wherein the analyses assesses the rate of change in the topography.

4. A method as recited in claim 2, wherein at least one of the analyses performed in step (b) includes measurements obtained during at least three separate visits.

5. A method as recited in claim 2, further includes assessing the rate of change in the topography based on linear regression of measurements.

6. A method as recited in claim 2, further including two or more follow-up visits wherein the follow-up visits are grouped into at least two periods and said analyses assesses the rate of change for each period respectively, and displays the rates on a common display.

7. A method of facilitating the identification of a statistically significant change in the thickness of the retinal nerve fiber layer (RNFL) in the eye of a patient comprising the steps of:
(a) obtaining optical measurements of the RNFL at least two different times;
(b) evaluating the results of the measurements to identify a change in thickness of the RNFL occurring between measurements, said evaluation including at least two different types of analyses, said two different analyses being selected from the group consisting of:
(i) a global analysis of RNFL thickness;
(ii) a regional analysis of RNFL thickness; and
(iii) a local analysis of RNFL thickness; and
(c) displaying or storing the results of the at least two different types of analyses.

8. A method as recited in claim 7, wherein the analyses assesses the rate of change of RNFL thickness.

9. A method as recited in claim 7, wherein the evaluation step (b) includes spatial registration of measurements obtained at different times.

10. A method as recited in claim 7, wherein results of the at least two different types of analyses are simultaneously displayed on a common display.

11. A method as recited in claim 7, wherein results of the at least two different types of analyses are combined to achieve more sensitive detection of RNFL change.

12. A method as recited in claim 7, wherein the evaluating step (b) includes three different analyses, at least one global analysis, at least one regional analysis and at least one local analysis, the results of which are simultaneously displayed.

13. A method as recited in claim 7, wherein the evaluating step (b) includes three different analyses, at least one global analysis, at least one regional analysis and at least one local analysis, the results of which are combined to achieve more sensitive detection of RNFL change.

14. A method as recited in claim 7, wherein the evaluating step (b) includes detecting blood vessels and excluding changes in blood vessel regions to improve accuracy of detection.

15. A method as recited in claim 7, wherein the evaluating step (b) includes detecting the optic nerve head (ONH) and excluding changes in ONH regions to improve accuracy of detection.

16. A method as recited in claim 7, wherein the evaluating step (b) includes confirming, within each type, the analysis results of a measurement with those of a consecutive measurement to improve accuracy of detection.

17. A method as recited in claim 7, wherein said local analysis includes one of a pixel comparison or a combination of a few adjacent pixels comparison derived from a two dimensional image.

18. A method as recited in claim 17, wherein the results of the local analysis are displayed by color coded overlays on the two dimensional image, a different two dimensional image, or a measurement data image, to indicate locations of reduced RNFL thickness.

19. A method as recited in claim 18, wherein the evaluation includes determining if selected image pixels exceed a first predetermined threshold and determining if there are a sufficient cluster of pixels exceeding a second predetermined threshold to indicate a reduced thickness RNFL.

20. A method as recited in claim 18, wherein the pixels corresponding to blood vessels are masked in the analysis to provide more accurate assessment of RNFL change.

21. A method as recited in claim 7, wherein the regional analysis includes mapping RNFL thickness in a region defined by a ring surrounding the optic nerve head of the eye.

22. A method as recited in claim 21, wherein the results of the regional analysis is displayed on a graphic illustrating a ring laid out over the quadrants of the eye and by color coding the regions within the ring that correspond to regions of reduced RNFL thickness.

23. A method as recited in the previous claim 22, wherein the evaluation includes determining if selected points within the ring exceed a first predetermined threshold and determining if there are a sufficient number of adjacent points that exceed a second predetermined threshold to indicate a reduced thickness RNFL.

24. A method as recited in the previous claim 22, wherein the points corresponding to blood vessels are masked in the analysis to provide more accurate assessment of RNFL change.

25. A method as recited in claim 7, wherein the regional analysis includes mapping RNFL thickness in a region defined by a segment of an annular ring surrounding the optic nerve head of the eye.

26. A method as recited in claim 7, wherein the global analysis includes calculating an average RNFL thickness over at least a portion of region defined by a ring surrounding the optic nerve head (ONH) of the eye or at least a portion of region defined by quadrants surrounding the ONH.

27. A method as recited in claim 7, wherein the global analysis includes calculating an average RNFL thickness across a region defined by a ring or a quadrant surrounding the optic nerve head of the eye.

28. A method as recited in claim 27, wherein the results are calculated and displayed as points of a trend line on a trend chart.

29. A method as recited in the previous claim 28, further including extrapolating the change in RNFL thickness and displaying the extrapolated results on the trend chart.

30. A method as recited in the previous claim 29, further including obtaining one or more additional measurements at a subsequent time, performing a global analysis of the results, generating information to generate a revised trend line and displaying both the original trend line and the new trend line on the trend chart.

31. A method as recited in claim 7, wherein the step of evaluating the results of the measurements to evaluate a change in thickness of the RNFL occurring between measurements is performed by directly comparing the measurements.

32. A method as recited in previous claim 31, wherein the evaluation of change requires at least three separate visits and the step of evaluating change is performed by directly comparing a later measurement with at least two earlier measurements to improve accuracy.

33. A method as recited in claim 7, wherein the step of evaluating the results of the measurements to evaluate a change in thickness of the RNFL occurring between measurements is performed by a statistical analysis of the trend of the measurements.

34. A method as recited in previous claim 33, wherein the step of evaluating the results of the measurements to evaluate a change in thickness of the RNFL occurring between measurements acquired with multiple systems is performed by the statistical analysis of the trend of the measurements accounting for measurement differences across systems.

35. A method as recited in claim 7, further includes determining if a reduction in RNFL is statistically significant and said reduction is noted on a display.

36. A method as recited in claim 7, further includes assessing the rate of RNFL change based on linear regression of measurements.

37. A method as recited in claim 7, further including two or more follow-up visits wherein the follow-up visits are grouped into at least two periods and said analyses assesses the rate of change for each period respectively, and displays the rates on a common display.

38. A method as recited in claim 7, wherein the test for a statistically significant change uses individual-base test-retest variability.

39. A method as recited in claim 7, wherein the test for a statistically significant change uses population-base test-retest variability.
